(19) United States
(12) Patent Application Publication
(10) Pub. No.: US 2011/0137261 A1
(43) Pub. Date: Jun. 9, 2011

(54) HYPODERMIC NEEDLE PROTECTOR

(75) Inventors: Jarrod GARBER, Johannesburg (ZA); Roy Miller, Johannesburg (ZA)

(73) Assignee: Hyposafe Investments (PTY) Ltd., Johannesburg (ZA)

(21) Appl. No.: 12/939,198

(22) Filed: Nov. 4, 2010

Related U.S. Application Data
(63) Continuation-in-part of application No. PCT/ZA2009/000036, filed on May 5, 2009.

(30) Foreign Application Priority Data
May 6, 2008 (ZA) 2008/02128

(51) Int. Cl. A61M 5/32

(52) U.S. Cl. 604/197

(57) ABSTRACT

A hypodermic needle protector (10) comprising: a sleeve having an open end (30) to accommodate a needle assembly (22,24) and a portion of a syringe cylinder (18) and a closed end (32) that has an opening (34) to receive a needle (22) of a needle assembly (22,24) there through a positioning member (44) within the sleeve, the positioning member (44) having an opening to engage the needle assembly (22,24) and the sleeve having at least one formation for releasably engaging the positioning member (44) and—a biasing member (48) situated in the sleeve between the positioning member and the closed end (32) of the sleeve. In use, counter-pressure exerted by the patent's skin against the closed end (32) causes the positioning member (44) to be rot

This is a Continuation-in-Part of PCT/ZA2009/000036 filed May 5, 2009, which claims priority of ZA 2008/02128, filed May 6, 2008.

The present invention relates to a hypodermic needle cover.

### BACKGROUND

A hypodermic syringe assembly comprises a cylinder with a nozzle at one end, a plunger and a needle assembly on the nozzle. The needle assembly comprises a needled secured to a plastics base that is releasably securable over the nozzle.

Needle stick injuries from exposed hypodermic needles are of great concern, particularly today in view of HIV/AIDS. Known needle assemblies are supplied with a cover that has a cross-section approximately the same as that of the base. The cover is removed for use of the needle and may be replaced after use. Alternatively the needle assembly is placed in a needle dispensing container. Until such time as the needle is covered or disposed of, the risk of needle stick injuries exists. Thus protectors or protection arrangements have been developed. Among such protectors are arrangements which withdraw the needle into the cylinder and covers that enclose at least the needle point automatically after use through biasing means.

The invention seeks to provide a hypodermic needle assembly protector that is simple and reliable.

### SUMMARY OF INVENTION

The invention provides a hypodermic needle protector comprising:
a sleeve having an open end to accommodate a needle assembly and a portion of a syringe cylinder and a closed end that has an opening to receive a needle of the needle assembly therethrough;
a positioning member within the sleeve, the positioning member having an opening to engage the needle assembly and the sleeve having at least one formation for releasably engaging the positioning member; and
a biasing member situated in the sleeve between the positioning member and the closed end of the sleeve.

Preferably the sleeve is movable in use, between a rest and an operative position. In the rest position the sleeve covers the needle and in the operative position the needle protrudes through the closed end beyond the sleeve.

Preferably the biasing means in integral with the positioning member.

Preferably the sleeve is releasably maintained in at least one of the rest and operative positions by the positioning member engaging the formation.

Preferably the positioning member is rotatable and movable along the length of the sleeve to engage and disengage the formations.

Preferably the sleeve comprises at least one longitudinal channel about which the positioning member is longitudinally movable. The protector may comprise a sleeve having at least two channels with the positioning member having a complimentary number and positioned channel engaging pins. The pins stabilize the movement of the positioning member within the sleeve, in use.

Preferably the positioning member is released from the operative position upon an activating force being applied against the closed end. In use, the protruding needle is inserted into the patient to a depth where the sleeve engages the patient's skin. The activating force being the counter-pressure exerted by the patient's skin against the closed end, causes the positioning member to be rotatably released from the formation thereby causing the sleeve to be released from the operative position and biased to the rest position. The biasing means causes the sleeve to cover the needle to the extent that the needle is withdrawn from the patient and eventually covers the entire needle once fully withdrawn from the patient. As such the sleeve automatically covers the exposed part of the needle after use thereby reducing the risk of contaminated needle-stick injuries.

Preferably a biasing means engaging step is located within the sleeve proximal to the closed end. The free-end of the biasing means engaging the step.

Preferably the face of the step, being the section which engages by the biasing means, is inclined and tapers away from the closed end of the sleeve. In use, the inclined face engages and causes the positioning member to be tensioned towards its rest position.

Preferably the closed end comprises an internal guide channel for guiding the passage of the needle through the opening.

Preferably the protector is tapered towards the closed end. This enables the user to more readily view the needle being inserted into the patient.

Preferably the guide channel has locking mechanism at its base through which the positioning member is rotationally guided and secured after use of the needle.

Conveniently the locking mechanism comprises a narrowed section about the base of the guide channel over which the channel engaging pins can pass and be received into a widened section. In use, the locking mechanism forms a unidirectional guide allowing the pins to pass through it and thereafter limits the return of pins, which locate in a recess, alternately a generally upright wall of the narrowed section.

Preferably a seal is located over the opening thereby preventing contamination and blunting of the needle prior to use.

In another embodiment of the invention there is provided a hypodermic needle protector comprising:
a sleeve having an open end to accommodate a needle assembly and at least a portion of a syringe cylinder extension and a closed end that has an opening to receive a needle of the needle assembly therethrough, the cylinder extension enabling the needle protector to be used on syringes of varying circumference and spout position;
a positioning member within the sleeve, the positioning member having an opening to engage the needle assembly and the sleeve having at least one formation for releasably engaging the positioning member;
a biasing member situated in the sleeve between the positioning member and the closed end of the sleeve;
the sleeve being movable along its longitudinal axis between a rest and an operative position; wherein the positioning member is released from the operative position upon a force being applied against the closed end.

Further features, variants and/or advantages of the invention will emerge from the following non-limiting description of an example of the invention made with reference to the accompanying drawings.

### BRIEF DESCRIPTION OF THE DRAWINGS

FIG. 1 shows a side view of a needle and syringe assembly with a cover of the invention;

FIG. 2 shows a sectioned side view of the needle and cover assembly of FIG. 1 through line A-A;

FIG. 3 shows a perspective top view of the needle and cover assembly of FIG. 1,

FIG. 4 shows a perspective side view of the needle and syringe assembly without the cover;

FIG. 5 shows a perspective side view of the needle assembly and cover apart from a syringe;

FIG. 6 shows a perspective top view of a spring assembly of the invention;

FIG. 7 shows a side view of a needle and syringe assembly with a cover of a second embodiment of the invention; and

FIG. 8 shows a sectioned side view of the needle and cover assembly of FIG. 7 through line 7-7.

### DETAILED DESCRIPTION OF THE INVENTION

FIG. 1 shows a side view of a needle cover or protector 10 over a needle assembly 12 that is attached to a syringe 14.

The syringe 14 comprises a plunger 16 and a cylinder 18 having a nozzle 20. The needle assembly comprises a needle 22 and a base 24, the base having a bottom lip 26. The needle assembly fits on to the nozzle.

The cover has an open end 30 dimensioned to receive the needle assembly and a portion of the syringe; and a closed end 32 having an opening 34 to receive the needle therethrough. The cover has an internal longitudinal groove formation 36 which has notches 38, 40 that extend laterally and downward away from the closed end and a laterally extending notch 42 at the open end. A positioning ring 44 having a pin 46 is contained inside the cover. The ring can rotate at the locking formations and is moveable longitudinally along the groove formation.

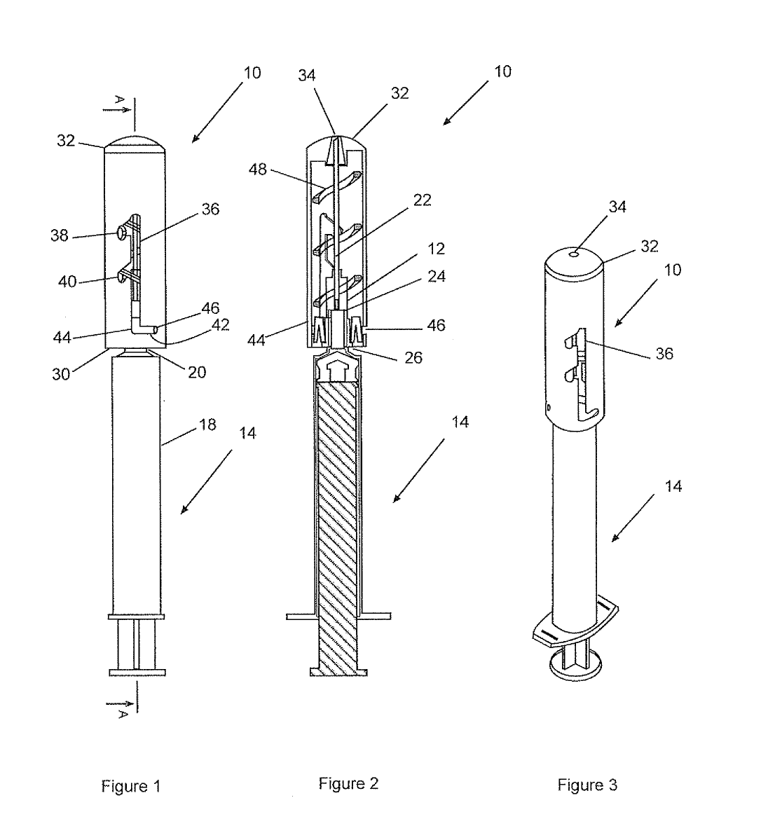

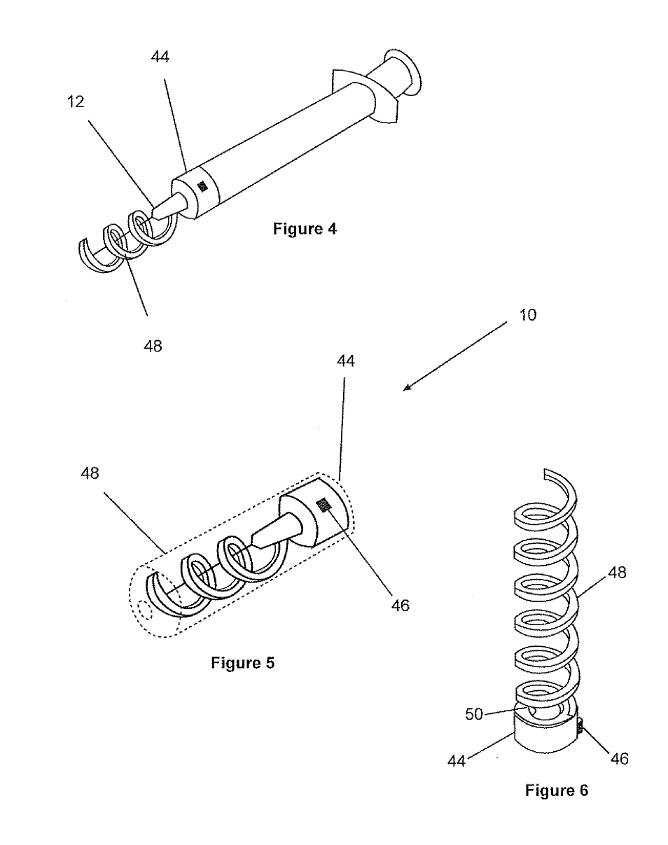

The ring firmly receives the needle base through an aperture 50 (as best shown in FIGS. 2 and 4). The lip limits movement of the ring over the base. The pin 46 guides the ring along the longitudinal groove and may be rotated for releasably engaging the ring with the locking formation. When the ring is engaged in one of the locking formations, the needle assembly is locked longitudinally in position relative to the cover. The ring may be of suitable plastics compound that may be as resilient or rigid as required.

A spring 48 is integral with the ring and extends from the top of the ring to the closed end of the cover. The spring biases the cover to its rest position. When the pin is not locked in one of the locking formations and the needle is inserted into the patient, the cover is biased to rest against the patient's skin. This results in the needle being automatically covered when it is withdrawn from the patient. The user is therefore not exposed to a blood contaminated needle at any stage.

In use, the counter-pressure exerted by the patient's skin causes the pin to be dislodged from the locking formation. The spring is tensioned to bias the pin away from the operative locking formations 30, 32, thereby ensuring the cover automatically covers the needle while simultaneously avoiding the operative locking formations. In addition, the tensioned spring causes the ring to be biased towards the rest locking formation which is opposite the operative formations. The accidental redeployment of the needle is prevented in the absence of rotating and aligning the pin with the longitudinal groove formation.

The operative locking formations enable the user to select a desired operative position. This position corresponds to the desired depth of the injection.

The diameter of the cover may vary to accommodate syringes of various diameters.

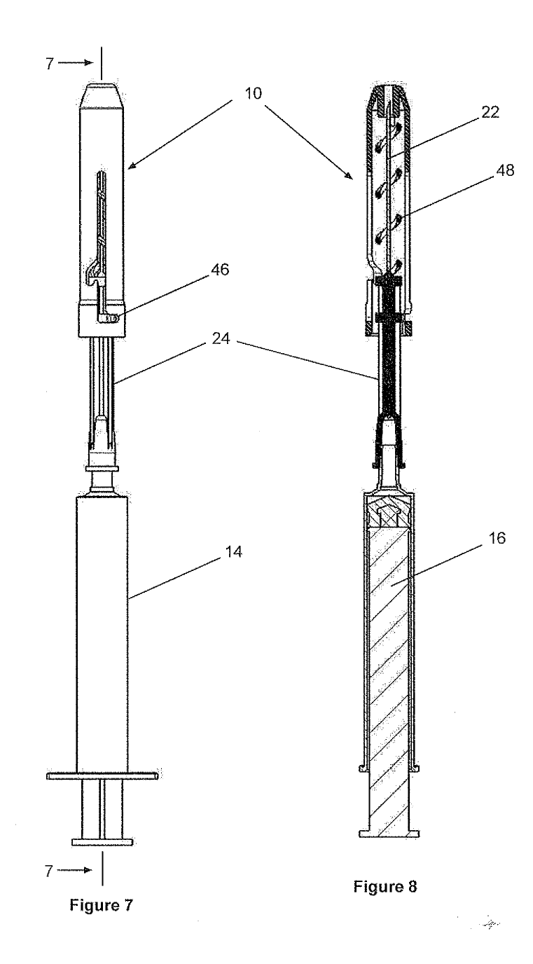

FIGS. 7 and 8 show a second embodiment of the invention wherein the base 24 is longitudinally elongate and serves to space the needle protector from the syringe nozzle. The cover of the needle protector thereby only moves longitudinally about the extended base and does not cover the syringe. In doing so, this embodiment may be used on any size or shape of syringe.

The invention is not limited to the precise details described above and shown in the drawings. Modifications may be made and other embodiments developed without departing from the spirit of the invention. For example the cover may have an internal step situated towards the closed end for engaging the free-end of the spring, the step having an inclined face for rotationally tensioning the spring to the rest locking position. The ring may have additional pins spaced along its circumference. Such rings are used in conjunction with complimentary longitudinal groove formations thereby providing added stability of the needle during use.

The claims and the summary of invention form an integral aspect of the description of the invention.

1. A hypodermic needle protector comprising:
a sleeve having an open end to accommodate a needle assembly and a portion of a syringe cylinder and a closed end that has an opening to receive a needle of the needle assembly therethrough;
a positioning member within the sleeve, the positioning member having an opening to engage the needle assembly and the sleeve having at least one formation for releasably engaging the positioning member;
a biasing member situated in the sleeve between the positioning member and the closed end of the sleeve;
the sleeve being movable along its longitudinal axis between a rest and an operative position; wherein the positioning member is released from the operative position upon a force being applied against the closed end.

2. The hypodermic needle protector as claimed in claim 1, wherein a biasing means engaging step is located within the sleeve proximal to the closed end.

3. The hypodermic needle protector as claimed in claim 2, wherein the face of the step, being the section which engages by the biasing means, is inclined and tapers away from the closed end of the sleeve.

4. The hypodermic needle protector as claimed in claim 3, wherein the closed end comprises an internal guide channel for guiding the passage of the needle through the opening.

5. The hypodermic needle protector as claimed in claim 1, wherein the biasing means in integral with the positioning member.

6. The hypodermic needle protector as claimed in claim 2, wherein the biasing means in integral with the positioning member.

7. The hypodermic needle protector as claimed in claim 1, wherein the sleeve is releasably maintained in at least one of the rest and operative positions by the positioning member engaging the formation.

8. The hypodermic needle protector as claimed in claim 1, wherein the positioning member is rotatable and movable along the longitudinal axis of the sleeve to engage and disengage the formations.

9. The hypodermic needle protector as claimed in claims 1, wherein the sleeve comprises at least one longitudinal channel about which the positioning member is longitudinally movable.

10. The hypodermic needle protector as claimed in claim 1, wherein the guide channel has locking mechanism at its base through which the positioning member is rotationally guided and secured.

11. The hypodermic needle protector as claimed in claim 10, wherein the locking mechanism comprises a narrowed section along the base of the guide channel over which the channel engaging pins can pass and be received into a widened section.

12. A hypodermic needle protector comprising:
a sleeve having an open end to accommodate a needle assembly and at least a portion of a syringe cylinder extension and a closed end that has an opening to receive a needle of the needle assembly therethrough;
a positioning member within the sleeve, the positioning member having an opening to engage the needle assembly and the sleeve having at least one formation for releasably engaging the positioning member;
a biasing member situated in the sleeve between the positioning member and the closed end of the sleeve;
the sleeve being movable along its longitudinal axis between a rest and an operative position; wherein the positioning member is released from the operative position upon a force being applied against the closed end.

13. The hypodermic needle protector as claimed in claim 12, wherein a biasing means engaging step is located within the sleeve proximal to the closed end.

14. The hypodermic needle protector as claimed in claim 13, wherein the face of the step, being the section which engages by the biasing means, is inclined and tapers away from the closed end of the sleeve.

15. The hypodermic needle protector as claimed in claim 12, wherein the biasing means in integral with the positioning member.

16. The hypodermic needle protector as claimed in claim 12, wherein the sleeve is releasably maintained in at least one of the rest and operative positions by the positioning member engaging the formation.

17. The hypodermic needle protector as claimed in claim 12, wherein the guide channel has locking mechanism at its base through which the positioning member is rotationally guided and secured.
